(19) United States
(12) Patent Application Publication
(10) Pub. No.: US 2016/0278998 A1
(43) Pub. Date: Sep. 29, 2016

(54) ABSORBER, ABSORBENT ARTICLE AND PROCESSES FOR PRODUCING THEREOF

(71) Applicant: LIVEDO CORPORATION, Shikokuchuo-shi, Ehime (JP)

(72) Inventors: Yuki Takahashi, Mima-gun (JP); Masatoshi Ikeuchi, Mima-gun (JP); Kenji Nakaoka, Osaka-shi (JP)

(73) Assignee: LIVEDO CORPORATION, Shikokuchuo-shi, Ehime (JP)

(21) Appl. No.: 15/034,043

(22) PCT Filed: Oct. 10, 2014
(86) PCT No.: PCT/JP2014/077129

§ 371 Date: May 3, 2016

(30) Foreign Application Priority Data
Nov. 5, 2013 (JP) 2013-229275

(51) Int. Cl. A61F 13/539; B29C 65/00; B29C 65/52; B32B 37/18; B32B 7/14; B32B 38/06; B32B 5/02; A61F 13/15; B32B 37/12

(52) U.S. Cl. CPC A61F 13/539; A61F 13/15739; A61F 13/15747; B29C 66/0324; B29C 66/45; B29C 66/7294; B29C 65/52; B32B 37/1292; B32B 7/14; B29C 66/0222; B32B 38/06; B32B 5/022; B32B 37/182; A61F 2013/53016

(57) ABSTRACT

An absorber (1) provided to an absorbent article, wherein the absorber (1) is formed by folding back an absorbent sheet body (2), which comprises a first sheet member (3) and a second sheet member (4) and contains an absorbent polymer (5) but does not contain a pulp fiber between the first sheet member (3) and the second sheet member (4), at both end parts (E) in the width direction, and an absorbent polymer is not disposed between the first sheet member (3) and the second sheet member (4) at the both end parts (E).

### TECHNICAL FIELD

The present invention relates to: an absorber used for an absorbent article such as an incontinence pad (including a light incontinence pad), a sanitary napkin and a disposable diaper; an absorbent article provided therewith; and processes for producing them.

### BACKGROUND ART

Conventionally, an absorbent article provided with an absorber (an absorbent sheet body) containing an absorbent polymer but does not containing a pulp fiber between sheet members is known. For example, Patent Literatures 1 to 4 disclose absorbent articles provided with such absorber.

### CITATION LIST

### Patent Literature

### Patent Literature 1

Japanese Unexamined Laid-open Patent Application Publication No. 2000-238161

### Patent Literature 2

Japanese Unexamined Laid-open Patent Application Publication No. 2004-275225

### Patent Literature 3

Japanese Unexamined Laid-open Patent Application Publication No. 2009-061230

### Patent Literature 4

Japanese Unexamined Laid-open Patent Application Publication No. 2013-042882

### SUMMARY OF INVENTION

### Technical Problem

In the above-explained absorbent sheet body, an absorbent polymer is disposed between sheet members; and since the absorbent polymer is a fine granular substance, it is difficult to fix all the absorbent polymers to the sheet members even when the absorbent polymers are boned to the sheet member by, for example, an adhesive to be fixed, and as a result, some of the absorbent polymer may fall from between the sheet members. In addition, since a pulp fibers is not provided between the sheet members, the absorbent polymer is not entangled with a pulp fiber to be held, whereby the absorbent polymer easily falls off. In this case, it is likely that the absorber does not exhibit an intended absorption performance or the absorbent polymer falling off the absorber deteriorates a wearing feeling.

The present invention has been achieved in view of the above circumstances, and the object of the present invention is to provide an absorber that is configured so that an absorbent polymer is disposed between sheet members but a pulp fiber is not disposed therebetween and the absorbent polymer is less likely to fall off, and an absorbent article provided with the absorber.

### Solution to Problem

The absorber of the present invention which solves the above problems is an absorber provided to an absorbent article and having a longitudinal direction and a width direction, wherein: the absorber is formed by folding back an absorbent sheet body, which comprises a first sheet member and a second sheet member and contains an absorbent polymer but does not contain a pulp fiber between the first sheet member and the second sheet member, at both end parts in the width direction; and an absorbent polymer is not disposed between the first sheet member and the second sheet member at the both end parts. Since the absorber of the present invention is formed by folding back the both end parts of such absorbent sheet body, the absorbent polymer is less likely to fall off the absorber in the width direction thereof and the absorber tends to exhibit an intended adsorption performance. In addition, the absorbent polymer is able to be placed even in the vicinity of end edges of the absorber in the width direction, thereby enabling enhancing the adsorption performance of the absorber.

It is preferred that the first sheet member and the second sheet member are joined to each other at the both end parts to form sealed portions. When the sealed portion is formed at the both end parts, the absorbent polymer does not easily fall off the absorber even if the absorbent polymer is moved to the both end parts from the center part.

It is preferred that the absorber is formed by folding back the absorbent sheet body in the width direction at the sealed portion. When the absorber is configured in this manner, the absorbent polymer is blocked to move from the center part to the both end parts on the occasion of folding back the both end parts of the absorbent sheet body to form the absorber, whereby the absorbent polymer is less likely to fall off the absorber.

It is preferred that the absorbent sheet body is embossed at the both end parts without the first sheet member and the second sheet member being melted, and the absorber is formed by folding back the absorbent sheet body in the width direction at the embossed part. When the both end parts of the absorbent sheet body are embossed in this manner, flexibility increases at the embossed part in the absorbent sheet body, that facilitates folding back of the both end parts in the absorbent sheet body.

It is preferred that the first sheet member is wider than the second sheet member in the absorbent sheet body, and the absorber is formed in that the absorbent sheet body is folded to the second sheet member side. When the width of the second sheet member which is located inside in folding the absorbent sheet body is made narrowed, it becomes easy to fold back the absorbent sheet body at the both end parts. In addition, the folded both end parts are made neat in the absorber.

It is preferred that the second sheet member is made of a nonwoven fabric whose constituent fibers orient in the longitudinal direction, and the absorber is formed in that the absorbent sheet body is folded to the second sheet member side. When the second sheet member which is located inside in folding the absorbent sheet body is made of a nonwoven fabric whose constituent fibers orient in the longitudinal direction, it becomes easy to fold back the absorbent sheet body in the width direction.

It is preferred that the absorbent sheet body has a center part positioned between the both end parts, the center part has a sealed portion where the first sheet member and the second sheet member are joined to each other and a non-sealed portion where the first sheet member and the second sheet member are not joined to each other, and the absorbent polymer is disposed in the non-sealed portion to form an absorption region. When the sealed portion is formed at the center part, the sealed portion functions as a diffusion passage of urine and the like, and the urine and the like which the absorber has received easily spreads through the sealed portion. As a result, urine and the like comes to be rapidly absorbed by the absorber.

The present invention also provides an absorbent article provided with the absorber of the present invention. In the absorbent article of the present invention, the absorber of the present invention is provided between a liquid-permeable top sheet and a liquid-impermeable a back sheet, wherein the absorber is formed in that the both end parts are folded to the second sheet member side, and the absorber is disposed so that the folded both end parts are located on the top sheet side or the back sheet side. When the absorber is disposed so that the folded both end parts are located on the top sheet side, the folded both end parts function to make urine and the like spread in the longitudinal direction of the absorber, whereby diffusivity of urine and the like is increased and leakage of urine and the like is less likely to be occurred owing to the folded both end parts. When the absorber is disposed so that the folded both end parts are located on the back sheet side, the absorber is liable to rapidly absorb urine and the like on the entire surface of the top sheet side thereof whereby improvement of absorption performance is expected.

The present invention also provides a process for producing an absorber, that enables producing the absorber of the present invention suitably. The production process of the present invention is a process for producing an absorber, that is formed by folding back an absorbent sheet body, which has both end parts in a width direction and a center part positioned therebetween, at the both end parts in the width direction, comprising the steps of: applying an adhesive to a first sheet member; disposing an absorbent polymer only at a center part of the first sheet member which the adhesive was applied to; laminating a second sheet member on the center part and both end parts of the first sheet member so that the absorbent polymer is disposed between the first sheet member and the second sheet member, thereby obtaining an absorbent sheet body; and folding back the both end parts of the absorbent sheet body to the second sheet member side.

In the step of folding back the both end parts of the absorbent sheet body, it is preferred that: the both end parts of the absorbent sheet body are folded back to the second sheet member side while suctioning the center part of the absorbent sheet body from the first sheet member side; or the both end parts of the absorbent sheet body are folded back to the second sheet member side while pressing a guide member against the absorbent sheet body along a longitudinal direction from the second sheet member side. When the both end parts of the absorbent sheet body is folded back to the second sheet member side in this manner, it becomes easy to fold back the both end parts at an exact position.

In the step of applying the adhesive to the first sheet member, it is preferred that the adhesive is applied to the center part and the both end parts of the first sheet member. By applying the adhesive to the both end parts of the first sheet member and then laminating the second sheet member on the first sheet member, the sealed portion can be formed easily at the both end parts of the absorbent sheet body.

It is preferred that the process for producing an absorber of the present invention further comprises the step of embossing the both end parts of the absorbent sheet body at a temperature lower than melting points of the first sheet member and the second sheet member, wherein the both end parts of the absorbent sheet body are folded back to the second sheet member side at the embossed part. By embossing the both end parts in this manner, stiffness of the embossed part is decreased and the both end parts are easily folded back at the embossed part.

When the absorber obtained in the above manner is disposed between a liquid-permeable top sheet and a liquid-impermeable back sheet, an absorbent article of the present invention can be obtained. The present invention also provides a process for producing an absorbent article, comprising the step of disposing the absorber obtained by the above production process between a liquid-permeable top sheet and a liquid-impermeable back sheet.

### Advantageous Effects of Invention

The absorber of the present invention is formed by folding back an absorbent sheet body, which contains an absorbent polymer but does not contain a pulp fiber between sheet members, at both end parts in the width direction, and an absorbent polymer is not disposed between the first sheet member and the second sheet member at the both end parts; and therefore, the absorbent polymer is less likely to fall off the absorber and the absorber easily exhibits an intended adsorption performance.

### BRIEF DESCRIPTION OF THE DRAWINGS

FIG. 1 represents an example of an absorbent sheet body that gives an absorber of the present invention and shows a partially cutout plan view of the absorbent sheet body.

FIG. 2 shows a plan view of the absorber formed by folding back both end parts of the absorbent sheet body shown in FIG. 1.

FIG. 3 shows a cross-sectional view along a line III-III of the absorber shown in FIG. 2.

FIG. 4 represents an example of an absorbent article provided with the absorber shown in FIGS. 2 and 3 and shows a plan view of the absorbent article.

FIG. 5 shows a first configuration example of a cross-sectional view along a line V-V of the absorbent article shown in FIG. 4.

FIG. 6 shows a second configuration example of a cross-sectional view along a line V-V of the absorbent article shown in FIG. 4.

FIG. 7 shows a schematic view of an example of a process for producing an absorber of the present invention.

### DESCRIPTION OF THE PREFERRED EMBODIMENTS

An absorber of the present invention is provided to an absorbent article such as an incontinence pad (including a light incontinence pad), a disposable diaper and a sanitary napkin. The absorber absorbs and fixes urine and the like which the absorbent article has been received, and is provided, for example, between a top sheet and a back sheet in the absorbent article.

The absorber has a longitudinal direction and a width direction. The “longitudinal direction” means a direction extending in a front-rear direction at a crotch of a wearer when a wearer wears the absorbent article provided with the absorber, and the “width direction” means a direction orthogonal to the longitudinal direction on the same plane as the absorber. A direction along a plane formed by the longitudinal direction and the width direction is defined as a plane direction. In addition, in the present invention, an “upper side” means a wearer's side in wearing the absorbent article and corresponds to a top sheet side against the absorber. A “lower side” means a side opposite to a wearer in wearing the absorbent article, that is an exterior side, and corresponds to a back sheet side against the absorber. A direction extending from the upper side to the lower side is defined as a perpendicular direction.

The absorber of the present invention is formed of an absorbent sheet body containing an absorbent polymer but not containing a pulp fiber between sheet members. The absorbent sheet body comprises a first sheet member and a second sheet member and contains an absorbent polymer but does not contain a pulp fiber between the first sheet member and the second sheet member. Thus, in the absorbent sheet body, an absorbent polymer is disposed between the first sheet member and the second sheet member, but a pulp fiber is not disposed between these sheet members. However, in the absorbent sheet body, presence of inevitably incorporated pulp fibers during the production is allowed. Since the absorbent sheet body contains an absorbent polymer but does not contain a pulp fiber between the first sheet member and the second sheet member, it can be formed to be thin while having a high absorption capacity.

The first sheet member and the second sheet member is preferably liquid-permeable, and examples of them include, for example, a nonwoven fabric formed from hydrophilic fibers such as cellulose, rayon and cotton; and a nonwoven fabric which is formed from hydrophobic fibers such as polyolefin (e.g., polypropylene, polyethylene), polyester (e.g., PET) and polyamide (e.g., nylon), and in which the hydrophobic fibers are hydrophilized with a surfactant on the surface thereof. As the first sheet member and the second sheet member, a woven fabric, a knitted fabric, a plastic film having pore or the like may be also used. Preferably, a nonwoven fabric is used for the first and second sheet members.

Examples of the absorbent polymer include polyacrylic acid-based absorbent polymers such as sodium polyacrylate; starch-based absorbent polymers such as a starch-acrylonitrile graft copolymer, a starch-acrylic acid graft copolymer, and a starch-acrylamide graft copolymer; and polyvinyl alcohol-based absorbent polymers such as a crosslinked polyvinyl alcohol. As the absorbent polymer, polyacrylic acid-based absorbent polymers such as sodium polyacrylate are preferably used, since they have a high liquid absorption capacity.

An absorbent polymer disposed between the first sheet member and the second sheet member is a fine granular substance, and therefore, even when the absorbent polymers are bonded to the sheet member by, for example, an adhesive to be fixed, it is difficult to fix all the absorbent polymers and some of the absorbent polymers may fall from between the sheet members. In addition, since a pulp fibers is not disposed between the first sheet member and the second sheet member, the absorbent polymer is not entangled with a pulp fiber to be held, whereby the absorbent polymer easily falls off. In this case, it is likely that the absorber does not exhibit an intended absorption performance or the absorbent polymer falling off the absorber deteriorates a wearing feeling.

Therefore, in the present invention, the absorber is formed by folding back the absorbent sheet body, which contains an absorbent polymer but does not contain a pulp fiber between the first sheet member and the second sheet member, at both end parts thereof in the width direction, and on this occasion, an absorbent polymer is not disposed at the both end parts between the first sheet member and the second sheet member, whereby the absorbent polymer is less likely to fall off the absorber.

The absorbent sheet body has the both end parts in the width direction and a center part positioned therebetween. The absorbent sheet body is folded back in the width direction at boundaries between the center part and the both end parts, and the folded part including an end edge, with respect to the width direction, of the absorbent sheet body is defined as the end part. The both end parts is composed of the end part of one side and that of the other side in the width direction. Lengths of the both end parts and the center part of the absorbent sheet body in the width direction is not particularly limited, and the center part is preferably formed so as to have 60% to 90% (more preferably 67% to 80%) of length of the absorbent sheet body in the width direction.

The absorber is formed by folding back the both end parts of the absorbent sheet body in the width direction, and the both end parts are disposed to overlap with an upper side or a lower side of the center part. For example, in the absorbent article in which the absorber is disposed between a top sheet and a back sheet, the both end parts may be folded to the top sheet side of the center part or may be folded to the back sheet side of the center part. Or, the one end part may be folded to the top sheet side and the other end part of that may be folded to the back sheet side among the both end parts; however, it is preferable that both of the end parts are folded to the top sheet side or the back sheet side. In the one or the other end part, the first sheet member and the second sheet member constituting each of the end parts are folded to the same side.

The folded both end parts may be joined (fixed) to the center part with an adhesive or the like. When the absorber is configured in this manner, folding back of the both end parts is maintained properly, whereby the absorbent polymer is less likely to fall off the absorber.

A shape (planar shape) of the absorbent sheet body is not particularly limited, and it is preferably a rectangular shape from a viewpoint of facilitating folding back of the both end parts of the absorbent sheet body. A shape (planar shape) of the absorber is also not particularly limited, and it is preferably a rectangular shape from a viewpoint of easily manufacturing.

At the center part, an absorbent polymer is disposed between the first sheet member and the second sheet member, and a pulp fiber is not disposed between them. The absorbent polymer may be disposed on an entire area of the center part or may be disposed on only a part of the center part. Since the absorber of the present invention is formed by folding back the both end parts of the absorbent sheet body, the absorbent polymer is able to be prevented from falling off the absorber from the end edge in the width direction even in the case where the absorbent polymer is placed at the end of the center part in the width direction. In addition, when the absorbent polymer is placed in this manner, absorption ability can be sufficiently exhibited even in the vicinity of the end edge of the absorber in the width direction, thereby enhancing the absorption performance of the absorber.

At the both end parts, an absorbent polymer is not disposed between the first sheet member and the second sheet member. Of course, a pulp fiber is also not disposed between the first sheet member and the second sheet member. Since an absorbent polymer is not disposed at the both end parts, loss of the absorbent polymer from the absorber is prevented.

It is preferred that the first sheet member and the second sheet member are joined to each other at the both end parts to form sealed portions. When the sealed portion is formed at the both end parts, the absorbent polymer does not easily fall off the absorber even if the absorbent polymer is moved from the center part to the both end parts. The sealed portion can be formed by joining the first sheet member and the second sheet member by an adhesive, heat-sealing (thermal fusion bonding) or ultrasonic bonding.

The sealed portion of the both end parts is preferably formed over an entire length of the absorber in the longitudinal direction. When the sealed portion is formed in this manner, the absorbent polymer is prevented from falling off the absorber in the width direction.

The sealed portion is preferably formed along a fold line at which the both end parts of the absorbent sheet body is folded back. Thus, it is preferred that the first sheet member and the second sheet member are joined to each other at the both end parts to form the sealed portions and the absorber is formed by folding back the absorbent sheet body in the width direction at the sealed portions. When the absorber is configured in this manner, the absorbent polymer is blocked to move from the center part to the both end parts on the occasion of folding back the both end parts of the absorbent sheet body to form the absorber, whereby the absorbent polymer is less likely to fall off the absorber.

The sealed portion of the both end parts is preferably formed by joining the first sheet member and the second sheet member with an adhesive. In this case, an adhesive layer described below may be provided at the both end parts and the first sheet member and the second sheet member may be joined to each other by the adhesive layer which is formed between the first sheet member and the second sheet member. The first sheet member and the second sheet member are preferably joined to each other with an intermittently applied adhesive, and by forming the sealed portion of the both end parts in this manner, flexibility of the first sheet member and the second sheet member at the both end parts is ensured and the absorbent sheet body is easily folded back at the sealed portion. As a method of intermittently applying the adhesive, an adhesive may be applied, for example, in a net-like pattern, a stripe pattern or a scattered-points pattern; and in the case of applying an adhesive in a net-like pattern, the adhesive may be applied by using a curtain spray method, a spiral coating method, an omega coating method or the like.

In view of facilitating folding of the absorbent sheet body at the both end parts, the absorbent sheet body is preferably formed that stiffness is lowered at the both end parts. In this respect, it is preferred that the absorbent sheet body is embossed at the both end parts without the first sheet member and the second sheet member being melted, and the both end parts are folded back at the embossed part. By embossing the both end parts of the absorbent sheet body in this manner, flexibility increases at the embossed part in the absorbent sheet body, that facilitates folding back of the both end parts in the absorbent sheet body.

For embossing the absorbent sheet body without melting the first sheet member and the second sheet member, the first sheet member may be embossed at a temperature lower than melting point of the first sheet member and/or the second sheet member may be embossed at a temperature lower than melting point of the second sheet member. Embossing may be subjected at least one of the first sheet member and the second sheet member. Embossing may be conducted in the state where the first sheet member and the second sheet member are laminated or may be conducted in the state where the first sheet member and the second sheet member are separated. Preferably, embossing is conducted in the state where the first sheet member and the second sheet member are laminated, and by embossing in this manner, it becomes easy to fold back the first sheet member and the second sheet member integrally. Thus, it is preferred that the first sheet member and the second sheet member are integrally embossed at the both end parts without being melted.

In view of facilitating folding of the absorbent sheet body at the both end parts, the sheet member located inside upon folding is preferably made of a nonwoven fabric whose constituent fibers orient in the longitudinal direction. For example, in the case that the absorber is formed by folding the both end parts of the absorbent sheet body to the second sheet member side, the second sheet member is preferably made of a nonwoven fabric whose constituent fibers orient in the longitudinal direction. When the second sheet member is made of a nonwoven fabric and its constituent fibers orient in the longitudinal direction, it becomes easy to fold back the absorbent sheet body in the width direction. Further, it is preferred that the first sheet member is also made of a nonwoven fabric whose constituent fibers orient in the longitudinal direction, and when both of the first sheet member and the second sheet member are made of a nonwoven fabric whose constituent fibers orient in the longitudinal direction, folding of the absorbent sheet body at the both end parts is made much easier.

Examples of the nonwoven fabric whose constituent fibers orient in one direction include a spunbonded nonwoven fabric, an air-through nonwoven fabric and a spun-lace nonwoven fabric. The orientation direction of constituent fibers of a nonwoven fabric can be determined by observing a surface of the nonwoven fabric with a microscope or the like. For example, in a spunbonded nonwoven fabric, the orientation direction of the constituent fibers is explained as follows. A spunbonded nonwoven fabric is formed by, for example, melting a polymer material, extruding it from a spinneret to be stretched, and collecting it on a conveyor belt or the like to form a web; and on this occasion, the web (fibers) collected on the conveyer belt is arranged along a traveling direction of the conveyer belt. Therefore, in this case, the web (fibers) comes to be oriented in the traveling direction (MD direction) of the conveyer belt. In an air-through nonwoven fabric, the orientation direction of the constituent fibers can be arranged by appropriately setting a collection method of raw short fibers in forming a fiber aggregate or a fiber-opening method in forming a web, upon manufacturing the nonwoven fabric. In a spun-lace nonwoven fabric, the orientation direction of the constituent fibers can be arranged by controlling flow of water in which short fibers are dispersed and depositing, upon manufacturing the nonwoven fabric.

In view of facilitating folding of the absorbent sheet body at the both end parts, it is also preferable that the absorber is configured as follows. That is, it is preferable that the first sheet member is wider than the second sheet member in the absorbent sheet body and the absorbent sheet body is folded to the second sheet member side, thereby forming the absorber. In this case, the absorber is preferably formed that the end edge of the first sheet member is located at an inner position, with respect to the width direction, of the end edge of the second sheet member in the folded both end parts. By narrowing the width of the second sheet member which is located inside in folding the absorbent sheet body, it becomes easy to fold back the absorbent sheet body at the both end parts. Conversely, by widening the first sheet member which is located outside in folding the absorbent sheet body, it becomes possible to cover the whole of the inside second sheet member with the outside first sheet member, thereby making the folded both end parts neat. In addition, in securing the folded both end parts to the center part with an adhesive or the like, it becomes easy to join the both end parts neatly by joining the first sheet member of the both end parts to the center part while covering the end edge, with respect to the width direction, of the second sheet member with the first sheet member.

In the case that the absorber is formed by folding the absorbent sheet body to the second sheet member side, it is also preferable that stiffness of the first sheet member is higher than that of the second sheet member. When the first sheet member and the second sheet member are configured in this manner, the both end parts of the absorbent sheet body are easily folded back to the second sheet member side.

Arrangement of the absorbent polymer at the center part is explained. It is preferred that the center part has a region where an absorbent polymer is disposed and a region where an absorbent polymer is not disposed. More preferably, the center part has a sealed portion where the first sheet member and the second sheet member are joined to each other and a non-sealed portion where the first sheet member and the second sheet member are not joined to each other, and an absorbent polymer is disposed in the non-sealed portion to form an absorption region. When the sealed portion is provided at the center part, the sealed portion functions as a diffusion passage of urine and the like, and the urine and the like which the absorber has received easily spreads through the sealed portion. As a result, urine and the like can be rapidly absorbed by the absorber.

The sealed portion is defined as a portion where the first sheet member and the second sheet member are joined together. In the center part, presence of an absorbent polymer at the sealed portion is allowed.

It is preferred that the sheet members are kept joined together at the sealed portion after the absorber absorbs. When the absorber absorbs, the absorbent polymer disposed between the sheet members swells and the sheet members may be possibly disjoined at the sealed portion. In this case, it is supposed that diffusivity of urine and the like in the absorber is deteriorated. Therefore, it is preferred that the sheet members are kept joined together at the sealed portion even after the absorber absorbs. In order that the sheet members are highly likely to be kept joined together at the sealed portion, it is preferred that the sheet members are bonded to each other with a rubber adhesive (e.g., a natural rubber type, a butyl rubber type, a polyisoprene type and others) or a styrene elastomer (e.g., styrene-isoprene-styrene block copolymer (SIS), styrene-butadiene-styrene block copolymer (SBS), styrene-ethylene-butadiene-styrene block copolymer (SEBS), styrene-ethylene-propylene-styrene block copolymer (SEPS) and others), or heat-sealed (thermal fusion bonded) or ultrasonic bonded to each other.

The non-sealed portion is defined as a portion where the first sheet member and the second sheet member are not joined together at the center part and corresponds to a portion other than the sealed portion. In the non-sealed portion, space is formed between the first sheet member and the second sheet member, and an absorbent polymer is disposed in this space to form the absorption region. In the center part, there may exist a non-sealed portion in which an absorbent polymer is not disposed in the space between the first sheet member and the second sheet member. The sealed portion is determined in a plan view of the absorbent sheet body.

The absorption region is formed in that an absorbent polymer is disposed between the first sheet member and the second sheet member. The absorption region functions to absorb and secure urine and the like which the absorber has received. It is preferred that a plurality of the absorption region are provided.

The absorption region is preferably provided at least in the vicinity of the end edge of the absorber in the width direction (that is, in the vicinity of the end edge, with respect to the width direction, of the center part). For example, it is preferred that the absorption region is provided at least in a region within 5 mm from the end edge of the absorber in the width direction. When the absorption region is provided in this manner, the absorber is able to exhibit absorption ability almost throughout the width direction thereof, thereby enhancing the absorption performance of the absorber. In the absorber of the present invention, even in the case where the absorption region is provided in the vicinity of the end edge of the absorber in the width direction, the absorbent polymer can be prevented from falling off the absorber.

An arrangement pattern of the absorption region is not particularly limited, and it is preferred that the absorption region is arranged in a stripe pattern or an island pattern. When the absorption region is provided in this manner, diffusivity of urine and the like in the absorber is increased and the overall absorption region provided in the absorber tends to contribute to absorption of urine and the like, properly.

In the case where the absorption region is arranged in a stripe pattern, it is preferred that at least two absorption regions are provided so as to extend in the longitudinal direction. In this case, it is preferred that at least one sealed portion extending in the longitudinal direction is provided so as to divide the absorption regions in the width direction. When the absorption region is arranged in a stripe pattern extending in the longitudinal direction, urine and the like easily spreads in the longitudinal direction of the absorber, whereby urine and the like tends to be rapidly absorbed by the absorber. In addition, it becomes possible to provide the absorption region in a relatively large area, thereby easily increasing the absorption capacity of the absorber.

In the case where the absorption region is arranged in an island pattern, it is preferred that at least two absorption regions are provided such that each of the absorption regions is surrounded by the sealed portion or the end edge of the absorber. When the absorption region is arranged in an island pattern, urine and the like easily spreads in the plane direction, whereby urine and the like tends to be rapidly absorbed by the absorber.

In the absorber of the present invention, the absorption region is preferably arranged in a stripe pattern extending in the longitudinal direction. When the absorption region is provided in this manner, the both end parts are easily folded back along the absorption region, and in the thus formed absorber, the absorption region comes to be placed in the vicinity of the end edge, with respect to the width direction, of the absorber.

An amount of the absorbent polymer disposed in the absorption region is not particularly limited and it is preferably 100 g/m² or larger, more preferably 150 g/m² or larger, and preferably 400 g/m² or smaller, more preferably 385 g/m² or smaller. When the absorbent polymer is disposed in the absorption region in the amount of 100 g/m² or larger, absorption capacity of the absorber tends to increases. Meanwhile, when the absorbent polymer is disposed in the absorption region in the amount of 400 g/m² or smaller, joining of the sheet members at the sealed portion tends to be maintained even after the absorbent polymer absorbs.

In the absorption region, the absorbent polymer is preferably fixed to the first sheet member and/or the second sheet member with an adhesive. That is, it is preferred that an adhesive is applied to the sheet member to form an adhesive layer and the absorbent polymer is fixed to the sheet member by the adhesive layer. The adhesive layer may be provided to at least one of the first sheet member and the second sheet member, and preferably, the adhesive layer is provided to both the first sheet member and the second sheet member. It is sufficient that at least a part of the absorbent polymer is fixed to the adhesive layer, and for example, the absorbent polymer in contact with the adhesive layer may be fixed to the adhesive layer. When the absorbent polymer is fixed to the sheet member with the adhesive layer, the absorbent polymer is less likely to move between the first sheet member and the second sheet member prior to the absorbent polymer absorbing, and the absorption capability of the absorber is easily ensured. Even after the absorbent polymer absorbs, the gelled absorbent polymer is less likely to move between the sheet members, and as a result, the absorbent polymer is less likely to form a lump to provide a feeling of discomfort to a wearer.

It is preferred that the adhesive layer does not inhibit absorbing and swelling of the absorbent polymer while fixing the absorbent polymer. In this respect, the adhesive layer is preferably formed in an intermittent pattern, and for example, the adhesive layer is preferably formed by applying an adhesive in a net-like pattern, a stripe pattern or a scattered-points pattern. For forming the adhesive layer in a net-like pattern, the adhesive may be applied by using a curtain spray method, a spiral coating method, an omega coating method or the like.

Examples of the adhesive used for the adhesive layer include, for example, rubber adhesives such as a natural rubber type, a butyl rubber type and a polyisoprene type; styrene elastomers such as styrene-isoprene-styrene block copolymer (SIS), styrene-butadiene-styrene block copolymer (SBS), styrene-ethylene-butadiene-styrene block copolymer (SEBS), and styrene-ethylene-propylene-styrene block copolymer (SEPS); ethylene-vinyl acetate copolymer (EVA); and polyolefin elastomers. These adhesives may be used either alone or as a combination of at least two of them. Among them, rubber adhesives or styrene elastomers are preferably used.

The sealed portion may be formed by the adhesive layer. In this case, the first sheet member and the second sheet member are joined together with the adhesive layer formed by applying an adhesive to the first layer and/or the second layer, thereby forming the sealed portion. The adhesive layer may be utilized for forming the sealed portion of the center part and may be utilized for forming the sealed portion of the both end parts. In the latter case, it is preferable in manufacturing that the adhesive layer is continuously applied from the center part to the both end parts.

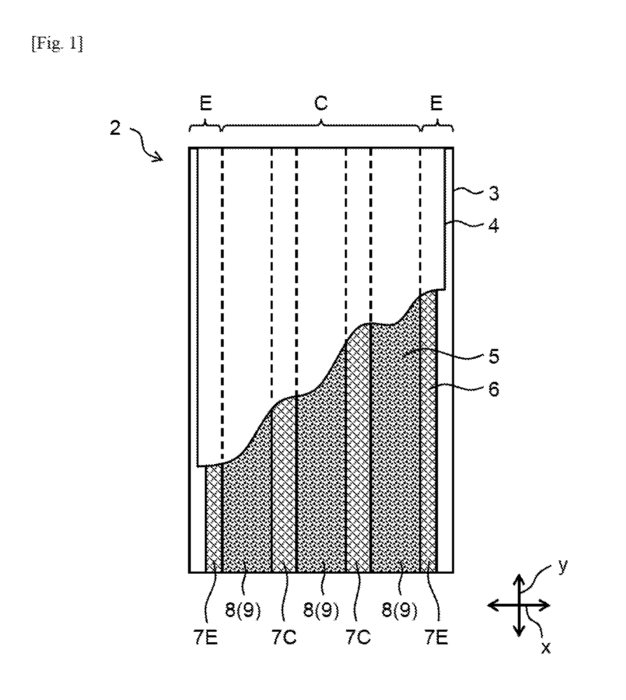

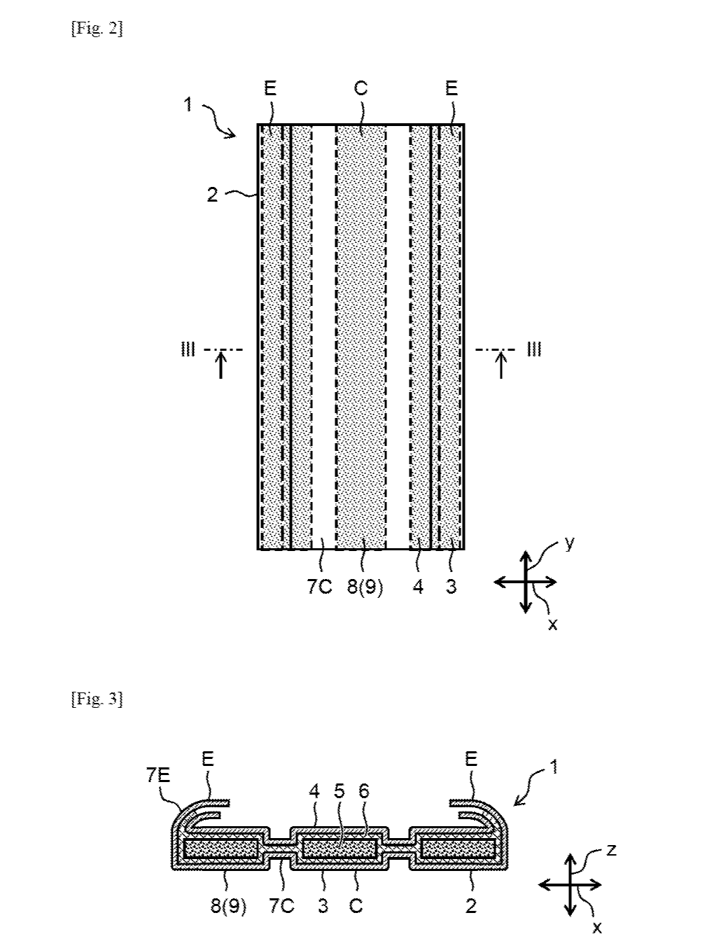

A configuration example of the absorber of the present invention is explained, referring to FIGS. 1 to 3. FIG. 1 shows a partially cutout plan view of the absorbent sheet body (that is, the absorber before being folded), FIG. 2 shows a plan view of the absorber formed by folding back both end parts, with respect to the width direction, of the absorbent sheet body shown in FIG. 1, and FIG. 3 shows a cross-sectional view along a line III-III of the absorber shown in FIG. 2. FIGS. 1 to 3 shows an embodiment in which the absorption regions are disposed in a stripe pattern. In the drawings, arrows x, y and z represent a width direction, a longitudinal direction and a perpendicular (thickness) direction, respectively.

An absorber is formed by folding back an absorbent sheet body 2 such as shown in FIG. 1 at both end parts E in the width direction x. The absorbent sheet body 2 has both end parts E in the width direction x and a center part C positioned therebetween. The absorbent sheet body 2 comprises a first sheet member 3 and a second sheet member 4, and contains an absorbent polymer 5 between the first sheet member 3 and the second sheet member 4 but does not contain a pulp fiber between the first sheet member 3 and the second sheet member 4. The absorbent polymer 5 is disposed at the center part C but is not disposed at both end parts E. The both end parts E of the absorbent sheet body 2 is folded inward in the width direction x, thereby forming the absorber 1 such as shown in FIGS. 2 and 3. Since the absorber 1 is formed such that the both end parts E are folded back in this manner, the absorbent polymer 5 is prevented from falling off the absorber 1 in the width direction x and the absorber 1 tends to exhibit a desired absorption performance. In addition, it is less likely that the absorbent polymer falling off the absorber 1 causes adverse effect to a wearer, for example. Furthermore, the absorbent polymer 5 is able to be disposed even in the vicinity of the end edge in the width direction x in the absorber 1, as shown in FIGS. 2 and 3.

An adhesive is applied to the first sheet member 3 and the second sheet member 4 to form adhesive layers 6, respectively, and the absorbent polymer 5 is fixed to the first sheet member 3 and the second sheet member 4 with the adhesive layers 6. At the center region C, the first sheet member 3 and the second sheet member 4 are joined to each other to form sealed portions 7C, and non-sealed portions 8 in which the first sheet member 3 and the second sheet member 4 are not joined to each other are formed. The absorbent polymer 5 is disposed in the non-sealed portions 8 to form absorption regions 9. The sealed portion 7C is formed by joining the first sheet member 3 and the second sheet member 4 with the adhesive layer 6. By providing the sealed portion 7C at the center part C, the sealed portion 7C functions as a diffusion passage of urine and the like, and rapid absorption of urine and the like can be realized.

At the both end parts E, the first sheet member 3 and the second sheet member 4 are joined to each other to form sealed portions 7E. The sealed portion 7E is formed by joining the first sheet member 3 and the second sheet member 4 with the adhesive layer 6. The absorber 1 is formed by folding back the absorbent sheet body 2 in the width direction x at the sealed portions 7E. When the sealed portions 7E are formed in this manner, the absorbent polymer 5 is blocked to move from the center part C to the both end parts 7E, whereby the absorbent polymer 5 is less likely to fall off the absorber 1.

In the absorbent sheet body 2, the first sheet member 3 is wider than the second sheet member 4, and the absorber 1 is formed by folding back the both end parts E of the absorbent sheet body 2 to the second sheet member 4 side. When the first sheet member 3 is formed wider than the second sheet member 4, it becomes easy to fold back the absorbent sheet body 2 at the both end parts E to the second sheet member 4 side. In addition, since the end edge of the first sheet member 3 is located at an inner position, with respect to the width direction x, of the end edge of the second sheet member 4 in the folded both end parts E, the absorber 1 is formed that the whole of the second sheet member 4 is covered with the first sheet member 3 in the both end parts E, whereby the appearance of the absorber 1 is made neat.

Next, an absorbent article of the present invention is explained. An absorbent article of the present invention is characterized in that the absorber of the present invention is provided, and the absorber of the present invention is disposed between a top sheet and a back sheet. Examples of the absorbent article include an incontinence pad (including a light incontinence pad), a disposable diaper and a sanitary napkin. In the absorbent article of the present invention, loss of the absorbent polymer from the absorber is less likely to occur and the absorber tends to exhibit an intended absorption performance. In addition, a wearing feeling is less likely to be deteriorated by the absorbent polymer which has fallen off the absorber.

A shape of the absorbent article is not particularly limited. In the case where the absorbent article is, for example, an incontinence pad or a sanitary napkin, examples of the shape of the absorbent article include a rectangular shape, an hourglass shape, a center nipped-in gourd shape, a battledore shape and others.

In the case where the absorbent article is a disposable diaper, the disposable diaper may be an open-type disposable diaper that is provided with a pair of fastening members on left and right sides thereof and is formed into a pants shape by using the fastening members in wearing, or the disposable diaper may be a pants-type disposable diaper that has a waist opening and a pair of leg openings.

The top sheet is a sheet which is located on a wearer's side in wearing the absorbent article and preferably liquid-permeable. The back sheet is a sheet which is located on an opposite side of the wearer, that is an exterior side, in wearing the absorbent article and preferably liquid-impermeable.

The top sheet and the back sheet are composed of, for example, a nonwoven fabric, a woven fabric, a knitted fabric, a plastic film, a laminate of a plastic film and a nonwoven fabric, or the like. Examples of the laminate include a laminate in which a sheet of a nonwoven fabric and a sheet of a plastic film are stacked, and a laminate in which a plastic film is interposed between nonwoven fabrics. In the case that a plastic film or a laminate including a plastic film is used for the top sheet, it is preferred that the plastic film has pores for allowing liquid to permeate through. The top sheet is preferably made of a nonwoven fabric. The back sheet is preferably made of a nonwoven fabric or a plastic film.

In the absorbent article of the present invention, the absorber is formed in that the both end parts are folded to the second sheet member side, and thus formed absorber may be disposed so that the folded both end parts are located on the top sheet side or the back sheet side relative to the center part. In the former case, when the absorber receives urine and the like, the folded both end parts function to make urine and the like spread in the longitudinal direction of the absorber, whereby diffusivity of urine and the like is increased, and lateral leakage of urine and the like is less likely to be occurred owing to the folded both end parts. Further, crotch of the wearer is less likely to strongly contact to the center part in which an absorbent polymer is contained by the presence of the folded both end parts, whereby the wearer is less likely to feel stiffness resulting from the absorbent polymer. In the latter case, the absorber is liable to rapidly absorb urine and the like on the entire surface of the top sheet side thereof, whereby improvement of absorption performance is expected.

In the absorbent article of the present invention, a plurality of the absorber may be stacked. In addition, an absorber other than the absorber of the present invention may be provided. For example, a supplemental absorber as another absorber may be provided below the absorber of the present invention. In this case, the supplemental absorber preferably contains a hydrophilic fiber (e.g., a pulp fiber, a cellulose fiber or the like) and an absorbent polymer, thereby improving leakage preventing effect of urine and the like from the back sheet side.

It is preferred that the absorbent article is provided with rising flaps on both sides in the width direction. Providing the rising flaps enables to prevent lateral leakage of urine and the like. The rising flap may be formed by, for example, joining side sheets which extend in the longitudinal direction to the top sheet on both sides in the width direction, and providing elastic members to inner ends, with respect to the width direction, of the side sheets. When the side sheet and the elastic member are provided in this manner, the inner ends of the side sheets rise toward wearer's skin due to a shrinkage force of the elastic member to form the rising flaps. The rising flap or the side sheet is preferably made of a liquid-impermeable plastic film, a liquid-impermeable nonwoven fabric, or the like.

The rising flaps are preferably provided so that rising base parts, that are rising origination points, overlap with the folded both end parts. In the absorber, portions where the center part and the folded both end parts are overlapped are particularly made rigid; and therefore, when the rising base parts of the rising flaps are provided so as to overlap with the folded both end parts, the rising flaps easily stand successfully. In addition, urine and the like excreted from the wearer comes to be led smoothly to the upper surface of the absorber and absorbed by the absorber suitably. Especially, in the absorbent article in which the absorber is formed in that the both end parts are folded to the second sheet member side and the absorber is disposed so that the second sheet member is located on the top sheet side, diffusivity of urine and the like in the longitudinal can be increased at the both end parts by providing the rising flaps such that the rising base parts overlap with the both end parts.

Elastic materials such as a polyurethane thread, a polyurethane film, a natural rubber, that are generally used for absorbent articles such as a disposable diaper, can be used for the elastic member provided to the rising flap. The elastic member is preferably fixed in a stretched state with a hot-melt adhesive. For example, a polyurethane thread having a fineness of 100 dtex to 2,500 dtex is stretched at a ratio of 1.1 to 5.0 times to be fixed. As a bonding means, a rubber hot-melt adhesive is preferably used. The forementioned ratio is defined to be 1.0 in unstretched state.

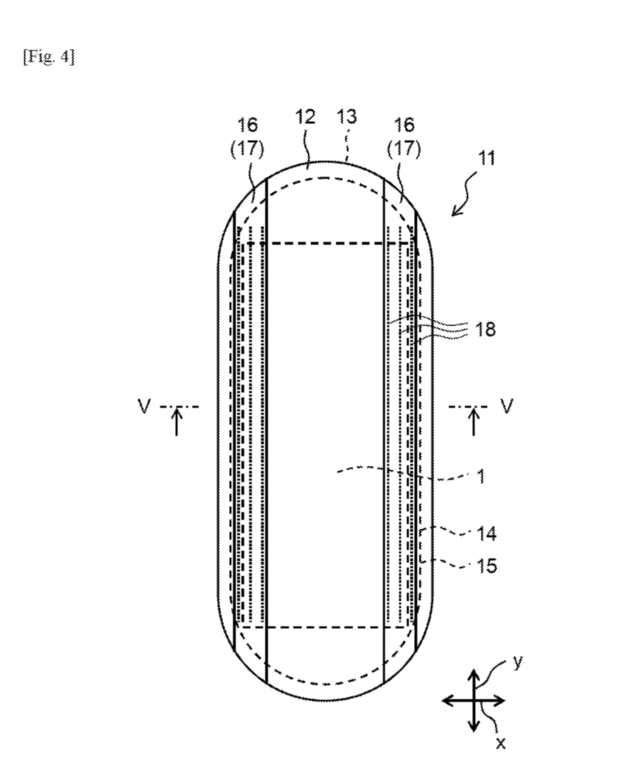

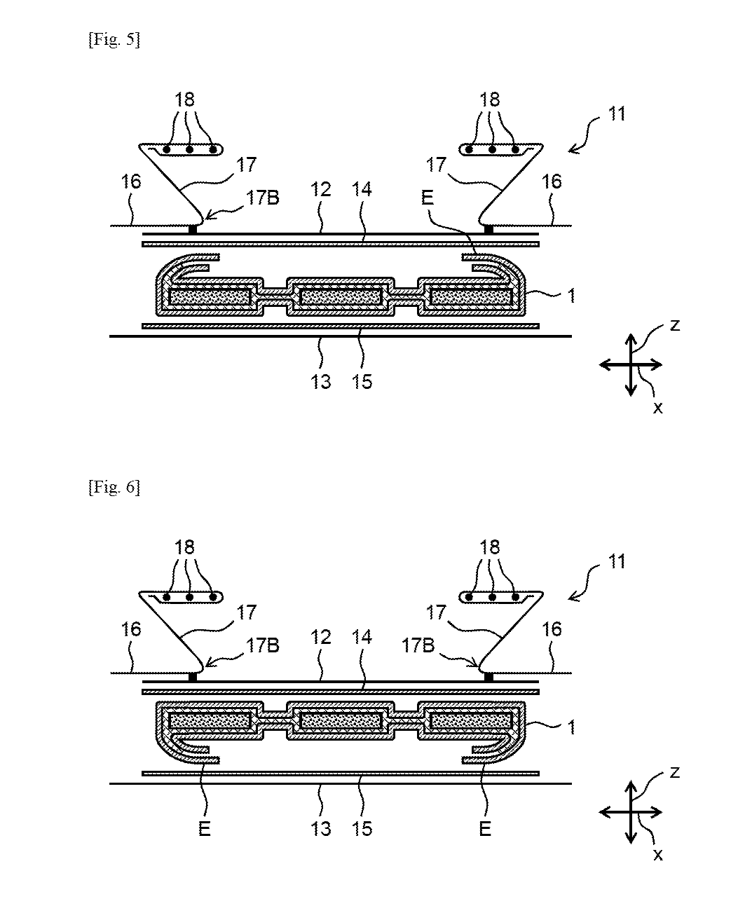

Examples of the absorbent article of the present invention is explained with an incontinence pad, referring to FIGS. 4 to 6. However, the absorbent article of the present invention is not limited to the embodiments shown in the drawings. FIGS. 4 to 6 represents an example of an absorbent article provided with the absorber shown in FIGS. 2 and 3. FIG. 4 shows a plan view of an incontinence pad as the absorbent article seen from a top sheet side. FIGS. 5 and 6 show cross-sectional views along a line V-V of the absorbent article shown in FIG. 4, and in FIG. 5 and FIG. 6, placement of the absorber in the vertical direction is different from each other.

An absorbent article 11 comprises a top sheet 12, a back sheet 13 and an absorber 1 disposed therebetween. The top sheet 12 is placed so as to face to a wearer's skin and allows a bodily fluid such as urine to permeate through. The bodily fluid which has passed through the top sheet 12 is accommodated in the absorber 1. The back sheet 13 is liquid-impermeable and prevents the bodily fluid from permeating outside.

In the absorber 1, the both end parts E are folded back, and the folded both end parts E are located on the top sheet 12 side in FIG. 5 whereas the folded both end parts E are located on the back sheet 13 side in FIG. 6. Urine and the like which the absorbent article 11 has been received is absorbed by the absorber 1 to be fixed. In the absorbent article 11 shown in FIG. 5, diffusivity of urine and the like in the longitudinal direction y is increased and the effect of preventing urine and the like from leaking in the width direction x is increased owing to the folded both end parts E. In the absorbent article 11 shown in FIG. 6, the absorber 1 is liable to rapidly absorb urine and the like on the entire surface of the top sheet 12 side thereof, thereby improving absorption performance.

It is preferred that an upper mat board 14 is provided between the top sheet 12 and the absorber 1. It is also preferred that a lower mat board 15 is provided between the back sheet 13 and the absorber 1. The upper mat board 14 and the lower mat board 15 are provided for promoting diffusion of a bodily fluid such as urine and for preventing the absorbent article 11 from loosing its shape. The upper mat board 14 is preferably liquid-permeable, and the lower mat board 15 may be liquid-permeable or liquid-impermeable. As the upper mat board 14 and the lower mat board 15, a tissue paper or a crepe paper may be used.

It is preferred that side sheets 16 extending in the longitudinal direction y are provided to the top sheet 12 on both sides in the width direction x. The side sheet 16 is joined to the top sheet 12 on both sides in the width direction x. In FIGS. 4 to 6, three rising elastic members 18 are disposed at an inner part, with respect to the width direction x, of the each side sheet 16. In using of the absorbent article 11, the inner part of the side sheet 16 rises toward a wearer's skin due to a shrinkage force of the rising elastic members 18, thereby forming a rising flap 17 and preventing excrement such as urine from leaking.

It is preferred that the rising flap 17 has a rising base part 17B of a rising origination point and the rising base parts 17B are positioned so as to overlap with the folded both end parts E. When the rising base parts 17B are disposed so as to overlap with the folded both end parts E, the rising flaps 17 easily stand successfully, and urine and the like excreted from the wearer is led smoothly to the upper surface of the absorber 1 to be absorbed by the absorber 1 suitably.

Next, a process for producing an absorber of the present invention is explained. A process for producing an absorber of the present invention, where the absorber is formed by folding back an absorbent sheet body, which has both end parts in a width direction and a center part positioned therebetween, at the both end parts in the width direction, comprises the steps of: applying an adhesive to a first sheet member (an adhesive application step); disposing an absorbent polymer only at a center part of the first sheet member which the adhesive was applied to (an absorbent polymer placement step); laminating a second sheet member on the center part and the both end parts of the first sheet member so that the absorbent polymer is disposed between the first sheet member and the second sheet member, thereby obtaining an absorbent sheet body (an absorbent sheet body-manufacturing step); and folding back the both end parts of the absorbent sheet body to the second sheet member side (a both end parts-folding step). According to the process for producing an absorber of the present invention, the absorber of the present invention is able to be manufactured easily.

In the adhesive application step, an adhesive is applied to a first sheet member. The adhesive is applied to at least the center part of the first sheet member. In the subsequent absorbent polymer placement step, an absorber polymer is disposed to the center part of the first sheet member which the adhesive was applied to, whereby the absorber polymer is secured to the first sheet member. In manufacturing the absorber continuously, the first sheet member is supplied in a continuous sheet and the adhesive is applied thereto. In this case, a direction orthogonal to the supplying direction of the first sheet member on the same plane as the first sheet member corresponds to a width direction.

In the adhesive application step, the adhesive is applied by, for example, a curtain spray method, a spiral coating method, an omega coating method or the like. By applying the adhesive in this manner, an adhesive layer formed in a net-like pattern can be provided. It is also preferred that the adhesive is applied in a stripe pattern or a scattered-points pattern.

In the adhesive application step, the adhesive may be applied to the center part and the both end parts of the first sheet member. By applying the adhesive to the both end parts of the first sheet member and laminating a second sheet member on the first sheet member in the subsequent absorbent sheet body-manufacturing step, the sealed portion can be formed easily at the both end parts of the absorbent sheet body.

In the absorbent polymer placement step, an absorbent polymer is disposed only at the center part of the first sheet member which the adhesive was applied to. In the absorbent polymer placement step, an absorbent polymer is disposed and fixed to the first sheet member. In the absorbent polymer placement step, it is preferred that absorbent polymers are disposed intermittently in the width direction, whereby the absorption region can be provided in a stripe pattern extending in the longitudinal direction and the sealed portion can be formed at the center part.

In the absorbent sheet body-manufacturing step, a second sheet member is laminated on the center part and the both end parts of the first sheet member so that the absorbent polymer is disposed between the first sheet member and the second sheet member, thereby obtaining an absorbent sheet body. In the absorbent sheet body-manufacturing step, the absorbent sheet body integrated by disposing an absorbent polymer between the first sheet member and the second sheet member is obtained. In the case of manufacturing the absorber continuously, the second sheet member is supplied in a continuous sheet and is laminated on the continuous first sheet member.

In the process for producing an absorber of the present invention, a step of applying an adhesive to the second sheet member may be provided prior to the absorbent sheet body-manufacturing step. In this case, the adhesive is preferably applied to at least the center part of the second sheet member, and further may be applied to the both end parts.

Also, a step of applying an adhesive to the first sheet member which the absorbent polymer has been disposed may be provided between the absorbent polymer placement step and the absorbent sheet body-manufacturing step. In this case, the adhesive is preferably applied to at least the absorbent polymer disposed on the first sheet member, and further may be applied onto a part of the first sheet member where an absorbent polymer is not disposed. The adhesive is preferably applied to at least the center part of the first sheet member, and further may be applied to the both end parts.

It is preferred that the second sheet member is narrower in the width direction than the first sheet member. When the second sheet member is narrower than the first sheet member, it becomes easy to fold the both end parts to the second sheet member side in the later both end parts-folding step and the resulting absorber is formed so that the appearance of the both end parts are made neat.

The absorbent sheet body obtained in the absorbent sheet body-manufacturing step is preferably embossed at the both end parts at a temperature lower than the melting points of the first sheet member and the second sheet member. That is, the process for producing an absorber of the present invention preferably further comprises the step of embossing the both end parts of the absorbent sheet body at a temperature lower than the melting points of the first sheet member and the second sheet member. By embossing the both end parts in this manner, stiffness of the embossed part is decreased and the both end parts are easily folded back at the embossed part in the later both end parts-folding step.

The absorbent sheet body obtained in the absorbent sheet body-manufacturing step may be heat-sealed (thermal fusion-bonded) or ultrasonic-bonded at the both end parts to form the sealed portions. In this case, an adhesive may not be applied to the both end parts of the first sheet member or the second sheet member for forming the sealed portion at the both end parts.

In the both end parts-folding step, the both end parts of the absorbent sheet body are folding back to the second sheet member side, thereby obtaining an absorber. In the process for producing an absorber of the present invention, it is preferred that: the both end parts of the absorbent sheet body are folded back to the second sheet member side while suctioning the center part of the absorbent sheet body from the first sheet member side (that is, an opposite side of the absorbent polymer-disposed side in the first sheet member); or pressing a guide member against the absorbent sheet body along the longitudinal direction from the second sheet member side.

When the center part of the absorbent sheet body is suctioned from the first sheet member side on the occasion of folding back the both end parts of the absorbent sheet body to the second sheet member side, it becomes easy to fold back the both end parts at an exact position even in the thin absorbent sheet body. In addition, the absorbent polymer is easily prevented from falling off in manufacturing the absorber. In this case, it is preferred that the both end parts of the absorbent sheet body are not suctioned from the first sheet member side. From the viewpoint of preventing the absorbent polymer from falling off in manufacturing, it is also preferable that the center part of the first sheet member is suctioned from an opposite side of the absorbent polymer-disposed side in the absorbent polymer placement step and/or the absorbent sheet body-manufacturing step.

In order to fold back the both end parts of the absorbent sheet body to the second sheet member side while suctioning the center part of the absorbent sheet body from the first sheet member side, the absorbent sheet body may be placed on a conveyer of which multiple holes for suctioning are formed at a part where the center part is positioned (for example, a conveyer of which the center part is formed in a mesh) and the both end parts of the absorbent sheet body may be lifted from the first sheet member side to fold back to the second sheet member side while the center part is suctioned from the first sheet member side to be secured, for example.

In the case of folding back the both end parts of the absorbent sheet body to the second sheet member side while pressing a guide member against the absorbent sheet body along the longitudinal direction from the second sheet member side, the guide member is applied to the boundary of the center part and the both end parts, and then the both end parts of the absorbent sheet body are lifted from the first sheet member side to fold back to the second sheet member side, whereby the both end parts of the absorbent sheet body are easily folded back at an exact position.

In the case of manufacturing the absorber continuously, the both end parts of the continuous body of the absorbent sheet body may be folded to the second sheet member side in the both end parts-folding step. In this case, an individually cut step may be provided after the both end parts-folding step, where the continuous body of the absorber is cut along a cutting line extending in the width direction in the individually cut step, thereby obtaining the individual absorber.

The absorber obtained in the above manner may be disposed between a liquid-permeable top sheet and a liquid-impermeable back sheet, thereby obtaining the absorbent article of the present invention. That is, a process for producing an absorbent article of the present invention comprises the step of disposing the absorber obtained by the process for producing an absorber of the present invention between a liquid-permeable top sheet and a liquid-impermeable back sheet. In the case of manufacturing the absorbent article continuously, the absorber (that may be cut individually) is disposed on a continuous body of the back sheet, a continuous body of the top sheet is disposed thereon, and the resultant is cut individually, thereby obtaining the individual absorbent article.

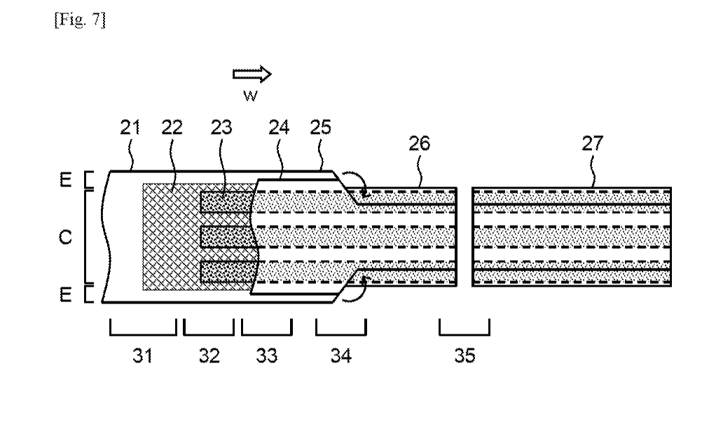

Next, an example of the process for producing an absorber of the present invention is explained, referring to FIG. 7. FIG. 7 shows an example of a process for continuously producing the absorber shown in FIGS. 2 and 3.

First, in an adhesive application step 31, a first sheet member 21 is supplied in a travelling direction w and an adhesive 22 is applied onto the first sheet member 21. In FIG. 7, the adhesive 22 is applied to an area from a center part C to both end parts E.

Then, in an absorbent polymer placement step 32, an absorbent polymer 23 is disposed only at the center part C of the first sheet member 21 which the adhesive 22 was applied to. In FIG. 7, the absorbent polymer 23 is arranged in a stripe pattern extending in the travelling direction w.

Then, in an absorbent sheet body-manufacturing step 33, a second sheet member 24 is laminated on the center part C and the both end parts E of the first sheet member 21 so that the absorbent polymer 23 is disposed between the first sheet member 21 and the second sheet member 24, thereby obtaining an absorbent sheet body 25. In FIG. 7, the first sheet member 21 is wider than the second sheet member 24, whereby the both end parts E of the absorbent sheet body 25 are easily folded back to the second sheet member 24 side in a subsequent both end parts-folding step.

At a part where the absorbent polymer 23 is not disposed and the adhesive 22 is exposed, the first sheet member 21 and the second sheet member 24 are joined to each other to form a sealed portion. In FIG. 7, the sealed portions are formed at the center part C and the both end parts E. Though it is not shown in the drawing, when an adhesive is applied to a surface of the second sheet member 24 that faces the absorbent polymer 23, the absorbent polymer 23 tends to be held stably between the first sheet member 21 and the second sheet member 24. Or, the absorbent polymer placement step 32 may be followed by a step of applying an adhesive onto the absorbent polymer 23.

In the both end parts-folding step 34, the both end parts E of the absorbent sheet body 25 are folded back to the second sheet member 24 side. On this occasion, the center part C of the absorbent sheet body 25 may be suctioned from the first sheet member 21 side or a guide member is pressed against the absorbent sheet body 25 along the travelling direction w from the second sheet member 24 side, whereby the both end parts E of the absorbent sheet body 24 are easily folded back to the second sheet member 24 side.

A continuous body 26 of an absorber obtained in the both end parts-folding step is cut in an individually cut step 35, thereby obtaining an individual absorber 27.

This application claims priority to Japanese Patent Application No. 2013-229275, filed on Nov. 5, 2013, the entire contents of which are incorporated by reference herein.

### REFERENCE SIGNS LIST

1, 27: an absorber
2, 25: an absorbent sheet body
3, 21: a first sheet member
4, 24: a second sheet member
5, 23: an absorbent polymer
C: a center part
E: both end parts
7C, 7E: sealed portions
8: a non-sealed portion
9: an absorption region
11: an absorbent article
12: a top sheet
13: a back sheet
17: a rising flap

1. An absorber provided to an absorbent article and having a longitudinal direction and a width direction, wherein
the absorber is formed by folding back an absorbent sheet body, which comprises a first sheet member and a second sheet member and contains an absorbent polymer but does not contain a pulp fiber between the first sheet member and the second sheet member, at both end parts in the width direction, and
an absorbent polymer is not disposed between the first sheet member and the second sheet member at the both end parts.

2. The absorber according to claim 1, wherein
the first sheet member and the second sheet member are joined to each other at the both end parts to form sealed portions.

3. The absorber according to claim 2, wherein
the absorber is formed by folding back the absorbent sheet body in the width direction at the sealed portion.

4. The absorber according to claim 1, wherein
the absorbent sheet body is embossed at the both end parts without the first sheet member and the second sheet member being melted, and
the absorber is formed by folding back the absorbent sheet body in the width direction at the embossed part.

5. The absorber according to claim 1, wherein
the first sheet member is wider than the second sheet member in the absorbent sheet body, and
the absorber is formed in that the absorbent sheet body is folded to the second sheet member side.

6. The absorber according to claim 1, wherein
the second sheet member is made of a nonwoven fabric whose constituent fibers orient in the longitudinal direction, and
the absorber is formed in that the absorbent sheet body is folded to the second sheet member side.

7. The absorber according to claim 1, wherein
the absorbent sheet body has a center part positioned between the both end parts,
the center part has a sealed portion where the first sheet member and the second sheet member are joined to each other and a non-sealed portion where the first sheet member and the second sheet member are not joined to each other, and
the absorbent polymer is disposed in the non-sealed portion to form an absorption region.

8. An absorbent article in which the absorber according to claim 1 is provided between a liquid-permeable top sheet and a liquid-impermeable a back sheet, wherein
the absorber is formed in that the both end parts are folded to the second sheet member side, and
the absorber is disposed so that the folded both end parts are located on the top sheet side.

9. An absorbent article in which the absorber according to claim 1 is provided between a liquid-permeable top sheet and a liquid-impermeable a back sheet, wherein
the absorber is formed in that the both end parts are folded to the second sheet member side, and
the absorber is disposed so that the folded both end parts are located on the back sheet side.

10. A process for producing an absorber, that is formed by folding back an absorbent sheet body, which has both end parts in a width direction and a center part positioned therebetween, at the both end parts in the width direction, comprising the steps of:
applying an adhesive to a first sheet member;
disposing an absorbent polymer only at a center part of the first sheet member which the adhesive was applied to;
laminating a second sheet member on the center part and both end parts of the first sheet member so that the absorbent polymer is disposed between the first sheet member and the second sheet member, thereby obtaining an absorbent sheet body; and
folding back the both end parts of the absorbent sheet body to the second sheet member side.

11. The process for producing an absorber according to claim 10, wherein
the both end parts of the absorbent sheet body are folded back to the second sheet member side while suctioning the center part of the absorbent sheet body from the first sheet member side in the step of folding back the both end parts of the absorbent sheet body.

12. The process for producing an absorber according to claim 10, wherein
the both end parts of the absorbent sheet body are folded back to the second sheet member side while pressing a guide member against the absorbent sheet body along a longitudinal direction from the second sheet member side in the step of folding back the both end parts of the absorbent sheet body.

13. The process for producing an absorber according to claim 10, wherein
the adhesive is applied to the center part and the both end parts of the first sheet member in the step of applying the adhesive to the first sheet member.

14. The process for producing an absorber according to claim 10, further comprising the step of embossing the both end parts of the absorbent sheet body at a temperature lower than melting points of the first sheet member and the second sheet member, wherein
the both end parts of the absorbent sheet body are folded back to the second sheet member side at the embossed part.

15. A process for producing an absorbent article, comprising the step of:
disposing the absorber obtained by the process according to claim 10 between a liquid-permeable top sheet and a liquid-impermeable back sheet.
